(19) United States
(12) Patent Application Publication
(10) Pub. No.: US 2005/0148669 A1
(43) Pub. Date: Jul. 7, 2005

(54) Amino acid esters as nutrient supplements and methods of use

(76) Inventor: Daniel Amato, Wellington, FL (US)

Correspondence Address: John P. Luther, Esq.;Newman & Newman, LLP, Suite 610, 505 Fifth Avenue South, Seattle, WA, 98104, US

(21) Appl. No.: 10/971,500

(22) Filed: Oct. 21, 2004

(51) Int. Cl. A61K 31/198; G06F 17/60

(52) U.S. Cl. 514/561; 705/2

(57) ABSTRACT

The present invention provides novel nutrient and food supplement amino acid ester compounds and compositions for human and animal consumption.

### FIELD OF THE INVENTION

The present invention relates to several novel amino acid ester compounds, formulations of these compounds, their use as nutrients or as food supplements and methods of conducting business thereby.

### BACKGROUND OF THE INVENTION

Copyright Notice 2004 Daniel Amato. All rights reserved. A portion of the disclosure of this patent application/patent document contains material which is subject to copyright protection. The copyright owner has no objection to the facsimile reproduction by anyone of the patent document or the patent disclosure as it appears in the United States Patent and Trademark office file and records.

Various esterified compounds for use as a dietary supplements and the like have been reported as providing a host of various advantages. For example, an esterified creatine product is said to assist in delivering a greater payload of cell volumizing creatine than other supplements, and is also said to display better absorption and less waste, for example, by penetrating muscle cells faster and more completely.

A vitamin C ascorbate/metabolite complex for use with animals is described as reducing discomfort associated with non-specific chronic inflammatory disorders and as being more rapidly absorbed from the gastrointestinal tract. This water and partially fat soluble complex is also said to cross membranes more efficiently in reaching higher cellular levels, and is also excreted at a slower rate than ordinary vitamin c. An esterified vitamin C version suitable for human consumption is sold as ascorbyl palmitate, and is a fat soluble form of ascorbic acid which is able to be stored in cell membranes until needed by the body. This compound is also said to be an effective free-radical-scavenging antioxidant effective for skin health and vitality.

In other examples, some esters of amino acids have shown efficacy in treating Alzheimer's disease. For example, U.S. Pat. No. 6,767,918 discloses a class of N-acetyl amino acid esters, such as alanine iso-butyl ester hydrochloride, N-(phenylacetyl)-D, L-alanine iso-butyl ester, N-(3-phenylpropionyl)-D, L-alanime-iso-butyl-ester, N-(3-methylpentanoyl)-L-alanine-iso-butyl-ester, N-[(4-chlorophenyl)acetyl]-L-alanine iso-butyl-ester, N-[3,4-dichloro-phenyl)acetyl]-L-alanine-iso-butyl-ester, and the like. Such compounds are prepared by the reaction of an amino acid of interest with a corresponding alcohol, such as the reaction of (D,L)-alanine or L-alanine with iso-butanol and catalyzed by thionyl chloride to produce (D,L)-alanine-iso-butyl ester hydrochloride. This compound is further transformed by reaction with phenylacetyl chloride to give the derivative N-(phenylacetyl)-D,L-alanine-iso-butyl-ester.

Other examples describe the preparation of amino acid-derivitized taxane ester compounds, such as the well known anti-neoplastic compound paclitaxel, by the condensation of the taxane and a protected amino acid. See, for example, U.S. Pat. Nos. 6,649,778 and 4,960,790. These derivitized taxane esters are known to provide enhanced water solubility for notoriously insoluble plant alkaloids. See also, for example, U.S. Pat. No. 4,943,579 which discloses certain amino acid derivatives of the anti-neoplastic plant alkaloid camptothecin as having enhanced water solubility. Additionally, the acylated ester of the amino acid acetyl-L-carnitine is reported for providing enhanced cellular energy metabolism and blood flow.

The introduction of amino acids to the body in a manner that would enhance their efficacy of action, such as derivitized amino acid esters for food and nutrient supplements, is highly desirable for a number of important reasons. Amino acids are the chemical units from which proteins are composed, and are oftentimes called building blocks. Proteins provide the structure for all living things and are the substances of which muscles, tendons, ligaments, organs, glands, nails, hair, vital body fluids, and bones are composed. Proteins are also responsible for the body's functions, such as immunoglobulins and the immune system, hormones, etc., all of which are responsible for such essential functions as oxygen transport and other life supporting activities.

Currently, several companies are offering various amino acids and amino-acid-containing compositions as food and nutrient supplements, but not in esterified or other derivitized form for enhanced bio-availability. For example, amino acid supplements are sold as Collatrim Capsules which are reported to build muscles and strengthen lean muscle mass even while a person sleeps. A Collatrim Plus product is said to support weight loss and to enhance the body's ability to burn fat, and to also tone muscles. Another Free Amino Acids product reportedly benefits individuals involved in strength and endurance training. L-carnitine is sold for its requirements in fatty acid transport, and its deficiency in humans is attributable to progressive muscle weakness and hypoglycemia. L-glutamine supplement is sold for its role in supplying the brain with energy, and L-lysine supplement is provided for its efficacy in strengthening circulation and in helping the immune system manufacture antibodies.

Additionally, the Ajinomoto Company is currently promoting a product called Amino Vital Water Charge tailored for use in marathons. This product contains fructose, salt, and a mixture of amino acids. The use of specific amino acids in sports beverages such as this product is thought to reduce muscle damage and fatigue during exercise, and to also enhance recovery after exercise.

In another example, U.S. Pat. No. 5,026,721 provides an amino acid nutritional supplement and regimen for enhancing physical performance. The described compositions comprise a mixture of biologically active amino acids which include at least glutamic acid, arginine, leucine, valine, and lysine. Optionally included are histidine, aspartic acid, threonine, serine, proline, glycine, alanine, cytstine, methionine, isoleucine, tyrosine, phenylalanine and tryptophan. The amino acid supplement is consumed prior to, or during exercise for performance enhancement, and is also described as an attractive alternative to other pharmaceutical performance enhancers, such as anabolic steroids thought by many to cause a host of complications. These supplements are said to result in a substantial enhancement of overall physical performance through sound nutrition.

U.S. Patent Publication No. 2003 0104107 describes an energy drink formula and method to provide enhanced athletic stamina and energy. These “sports” drinks contain a variety of ingredients, inclusive of, for example, a carbohydrate source, a mineral source, such as calcium, sodium, potassium, magnesium and zinc, a vitamin source, such as vitamin B¹², pantothenic acid, vitamin C, vitamin E and a mixture of branched and straight chain amino acids. The amino acid component is said to be responsible for proper brain functioning, immune system functioning and cellular replication, for proper functioning of the central nervous system, and in energy production during exercise, especially in exerting muscles. Other attributes are, inter alia, help with spare muscle tissue and the promotion of muscle tissue, the promotion of muscle growth and healing. The sports drink, as currently used by University football teams, with its combination of straight chain and branched chain amino acids is thought to supplement glucose for energy production in muscle tissue.

An additional nutritional composition is described in U.S. Pat. No. 6,541,026 which comprises, among other things, natural amino acids in amounts effective to help reduce body fat in humans, aloe vera, collagen, garcinia, cambogia, chromium polynicotinate, conjugated linolic acid and fiber. This nutritional composition is useful for reducing body fat, promoting the formation and protection of human lean muscle tissue and enhancing the human body's use and absorption of nutrients.

In a further example of the use of amino acids in human consumption, U.S. Patent Publication No. 20020155170 describes nutrient supplements and methods effective for treating autism and for preventing the onset of autism. These compositions include a mixture of minerals, vitamins, amino acids and agents capable of delivering cysteine in vivo. The supplements are also capable of promoting metallothioneins, and for exhibiting a chelating effect by promoting the removal of excess metals from the bloodstream. Non-essential amino acids, such as taurine, are preferred for the removal of excess metals, and those amino acids preferred for promoting metallothioneins in a person include serine, lysine, alanine, glycine, threonine, praline, aspartic acid, asparagine, glutamic acid, methionine, glutamine, isoleucine, valine and cysteine.

Additionally, while not specifically disclosed to be useful as nutrient supplements in humans, Lebmann et al., Z. Naturforsch. 53b, 981-990 (1998) has reported the reactions of benzoyl isothiocyanate with amino acid ethyl esters, such as N-butyl glycine ethyl ester or (S)-(−)-proline ethyl ester, which produces N-benzoylthiocarbamoyl amino acid ethyl esters effective as chelate ligands for transition metal ions.

Other reported results have suggested that L-valine is a desirable amino acid for the esterification of poorly permeable drugs to enhance their oral bio-availability in targeting the intestinal peptide transporter PEPTI in epithelia, which is thought to play an important role in the absorption of small peptides and peptide-like drugs.

As one may see from the above discussion, given the vital roles amino acids play in the body and the several methods and compositions for delivery of amino acids as supplements, It would be highly beneficial to provide food and nutrient supplements and other compositions for human use and consumption which comprise one or more, or an array, of amino acids in derivatized form, such as esterified amino acids, for their enhanced deliverability and bio-availability, and concomitantly enhanced efficacy in a variety of indications and body functions.

It would also be highly beneficial to provide such food and nutrient supplements comprising esterified amino acids and/or derivitized amino acids for animal use and consumption.

It would be still further beneficial to provide novel methods of conducting business comprising the design, manufacture, marketing, distribution and sale of such products, and/or the use of such products in the formation and operation of a business entity, in the development of business goodwill, and the generation of valuable trademark rights as a source identifier of such products and methods.

### SUMMARY OF THE INVENTION

In accordance with that above, the present invention provides novel nutrient and food supplement amino acid ester compounds and compositions for human and animal consumption, orally deployable formulations and other formulations of such compounds and compositions and methods of treating humans and animals with such novel compounds and compositions. This invention also provides novel methods of conducting business activities relating to the designing, manufacture, packaging, marketing, distributing, selling, licensing and leasing, leasing and other business functions associated with the use of the novel nutrient supplements and compositions thereof.

The invention is more fully understood with reference to the following Detailed Discussion of Preferred Embodiments.

### DETAILED DISCUSSION OF PREFERRED EMBODIMENTS

All patent references, published patent applications and literature references referred to or cited herein are expressly incorporated by reference to the same extent as if each were specifically and individually indicated to be incorporated by reference. Any inconsistency between these publications and the present disclosure is intended to and shall be resolved in favor of the present disclosure.

Throughout this specification and the claims which follow, unless the context requires otherwise, the word “comprise” and variations such as “comprises” and “comprising” will be understood to imply the inclusion of a stated compound, bio-active agent, carrier, vehicle, adjuvant or excipient or step or group thereof but not the exclusion of any other compound, bio-active agent, carrier, vehicle, adjuvant, or excipient or step or group thereof.

The language “in combination with” another agent, be it, for example, a bio-active agent, carrier, vehicle, adjuvant or excipient (collectively “agent(s)”) includes co-administration of the compound or compounds of the invention (“compound(s)”) and the agent, administration of the compounds(s) of the invention first, followed by the agent(s) and by administration of the agent(s) first, followed by the compound(s) of the invention.

Amino acids are both carboxylic acids and amines, which compounds consist of an alpha carbon atom to which is attached a hydrogen atom, an amino group, a carboxyl group, and an “R” group which structure determines the identity of the amino acid and the compound's unique physical and chemical properties. As known, the chemical nature of R will for the most part determine the particular amino acids' aqueous solubility characteristics and whether the molecule is changed.

There are in excess of one-hundred known amino acids, with each having a different R group, and which are obtained from both plant and animal sources. Twenty or so amino acids have been identified as making up the proteins of all living organisms, and thus are commonly referred to as building blocks. Because proteins provide the structure for living organisms, and amino acids are an essential component of protein, amino acids are very important to maintain the health of an organism. Proteins make up a majority of a person's body weight, second only to water, and are responsible for the composition of the body's structural components, such as muscles, tendons and ligaments, and other body parts such as organs, glands, nails, hair, vital body fluids, bones, in addition to functioning as enzymes and hormones in the regulation of all bodily processes. Some twenty-eight known amino acids link together in various combinations in chains to form proteins, with specific arrangements producing proteins or unique structure, conformation and highly specific and unique function and reactivity, such as specialized enzymes and the like.

In general, such specific amino-acid linking arrangements, the frequency of their production, etc., are determined by the genetic code, or more specifically, the nature of a person's or organism's DNA make up. Within each cell of an organism are strands of deoxyribonucleic acid (DNA) which represents instructions as to how a cell is to fabricate its protein(s) from amino acids. The DNA instructions, commonly called the DNA code, consist of strings of 64 alphabet characters which specify a particular amino acids' placement in building a protein's chain structure by a group of three characters (three base pairs), know as codons. Single characters of the codons, or single base pairs, are strung together in various combinations in a twisted helix to form a DNA strand (the double helix).

In protein manufacture by stringing amino acids together, DNA blueprint instructions are translated into enzyme synthesis, which enzymes in turn enable and control molecular synthesis. Reading amino acid sequence instructions from DNA codon instructions and for transfer of such information into protein synthesis is accomplished in the cell by ribonucleic acid (RNA), or more specifically transfer RNA (tRNA). Protein construction via use of such information is performed in ribosomes, or protein synthesis centers. Generally, depending upon need, or some chemical signal, ribosomes develop a tRNA template for the protein to be manufactured and draw free amino acids from the cellular pool into the called-for orderly sequence. The ordered amino acids are then linked by enzymatic action in the construction of a protein of a particular nature. Short chain lengths of amino acids are referred to as peptides, with longer chains referred to as polypeptides. The longest chains comprising about 300 or more amino acids form unique shapes and specialized 3-dimensional information necessary for specialized function. Such a protein's shape is in effect information which determines its biological role.

As may be seen, an error in DNA coding may result in a downstream manufacturing error, such as in a pivotal enzyme's chemical structure, and thus a defect in its shape and information, with the net result being an improperly functioning protein, such as may be necessary, for example, for proper immune response, cell repair, cell growth and the like. DNA coding errors and/or an abnormal rate of protein synthesis may sometimes be the result of an insufficient or even inefficient supply of amino acids.

The supply of amino acids to the body is determined by the particular nature of each amino acid. Certain amino acids which are not produced by the organs and glands of the body are called essential amino acids, and must be obtained through ingesting of food material into the body. These amino acids include histidine, isoleucine, leucine, lysine, methionine, phenyl-alanine, threonine, tryptophan and valine. The body cannot synthesize these molecules, which must be obtained from the diet. The remaining amino acids from the group found in protein synthesis are referred to as non-essential. This designation does not mean that such compounds are unnecessary, but rather that they do not have to be supplied by diet and are synthesized by the body from other amino acids.

The process of creating proteins, enzymes, hormones, tissue and the like is an ongoing process, with proteins being recycled and broken into amino acids, usually by proteins, and which are then assembled into other proteins, enzymes, etc. as the body's bio-chemical processes and functions call for. Thus, the body must always have a ready supply of proteins and other raw material in the form of proteins, carbohydrates, vitamins and the like, needed to operate its protein manufacturing machinery and operations. In this light, it can be seen how the nature of one's diet is so important. To this end an industry of providing sources of amino acids, proteins and amino acid supplements has evolved to aid one's diet in this manner.

Diet sources of amino acids are usually categorized into two different groups, which depend upon the particular amino acids they contain. One group is referred to as “complete proteins” which contain amounts of the essential amino acids. Sources of these proteins may be, for example, meat, fish, poultry, cheese, eggs and milk. The other group, referred to as “incomplete proteins”, contain less than all of the nine essential amino acids, and such proteins may be found, for example, in grains, legumes and leafy green vegetables.

While the body needs a full range of both essential and non-essential amino acids, it is not necessary to always obtain them from the above sources. It may be desirable, or even necessary in some, to avoid animal flesh sources and their usually high fat content, and associated hormones, antibiotics, and possible bacterial contamination. Fish sources may not be desirable for others due, again, to possible microbial contamination, and heavy metal contamination in some species, such as tuna fish and swordfish. Thus supplementation diets have been devised, such as for vegetarians, who wish to avoid such food/protein sources, and which combine partial complete protein diets with other supplements to supply adequate amounts of essential amino acids, such as a diet of mixing a legume with a grain, for example, brown rice, or with a tuber, to provide desired amounts of minerals and vitamins with a complete amino acid mixture.

It is generally thought that for protein synthesis to occur, all the required amino acids in adequate amounts must be present at the same time. However, proper diets are not always possible, and even when possible many people would like to limit caloric intake to check unwanted weight gain, while others would like to channel protein and amino acid sources to selected use, such as energy enhancement, and muscle development. Amino acid supplementation of foods and animal feeds has been shown to bring about many beneficial results, with the attainment of a favorably balanced diet and other desired results, such as energy enhancement, muscle rebuilding and the like.

It is also known, however, that a certain percentage of protein-bearing food is destroyed in the digestive tract, and a high percentage of amino acids are broken down and destroyed by the liver. Thus, for example, an oral intake of a high concentration of an amino acid supplement containing an array of essential and non-essential amino acids may be seen as wasteful, as very few will be actually used by the body as protein building blocks for enzymes, etc.

In one aspect, and in several preferred embodiments, the present invention helps to avoid such premature destruction by providing esterified amino acids as nutrient supplements, some of which are of an enhanced lipophillic nature and more fat soluble for better absorption into the body with less waste before digestive tract destruction and liver degradation.

Therefore in accordance with the present invention, there are provided novel nutrient supplement amino acid esters, formulations of these esters and methods of nutritional supplementation of humans and animals with such esters and formulations.

Generally, amino acid esters useful in the present invention are of the formula: 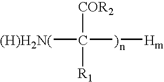
wherein R₁ is a residue of a biologically significant amino acid and/or naturally occurring amino acid selected from arginine, glycine, alanine, beta-alanine, valine, isovaline, norvaline, leucine, isoleucine, norleucine, alloisoleucine, phenylalanine, proline, serine, homoserine, allothreonine, threonine, tyrosine, cysteine, homocysteine, cystine, methionine, lysine, histidine, tryptophan, aspartic acid, glutamic acid, asparagine, glutamine, taurine, sarcosine, ornithine, citrulline, aminobutyric acid, isobutyric acid, aminoisobutyric acid, amino-n-butyric acid, allo-isoleucine, pyroglutamic acid, thiaproline, seleno-L-methionine, 4-hydroxyproline, 5-hydroxyproline, aminoadipic acid, cadaverine (15-diaminopontane), 4-amino-benzoic acid, x-aminopimelic acid, 2,4-diamino-n-butyric acid, gylcine-glycine, ornithine, glycine-proline, threonine-aspartic acid, hydroxylysine, diaminopimelic acid, praline-hydroxyproline, lysine-alanine, dopamine, cystathionine, 3,4-dihydroxy-phenylalanie, argene-succinic acid, gamma amino butyric acid, statine, carnitine, ethionine, serotonin, peptides, oligo-peptides and proteins thereof, other naturally occurring amino acids, and
where R₂ comprises one or more of alkyl, cycloalkyl, alkyl, substituted alkyl, cycloalkyl, substituted cycloalkyl, heterocyclic, substituted heterocyclic, alkenyl, substituted alkenyl, alkynl, substituted alkynyl, aryl, substituted aryl, heteroaryl, substituted hereroaryl, alkylaryl, substituted alkylaryl, arylalkyl, substituted arylalkenyl, substituted arylalkenyl, arylalkynl, substituted arylalkynl, aroyl, substituted aroyl, acyl, substituted acyl, or the like and any and all isomers and teutomeric forms thereof, or the two R groups can cooperate to form a 5-, 6- or 7-membered ring including N and the two R groups, or either of the R groups is a divalent moiety selected from the group consisting of alkylene, substituted alkylene, oxyalklene, substituted oxyalkylene, alkenylene, substituted alkenylene, arylene, substituted arylene, alkarylene, substituted alkarylene, aralkylene and substituted aralkylene, wherein said divalent moiety serves as the same substituent for two dithiocarbamate structures, thereby linking said structures together so as to form a bis(dithiocarbamate) species; and where n is an integer from 1 to 20 and m is an integer.

As used herein the term “biologically significant amino acid” means any amino acid known in the art to be bio-active in mammals to any degree, or which displays any amount of efficacy for any indication in humans and animals. See, for example, Chemical Abstracts, Naming and Indexing of Chemical substances (1987).

The term “oligo-peptide” as used herein refers to small chain amino acid combinations, such as, for instance, synthetic hormone growth releasing peptides (GHRPs), of which an example is ghrelin, a 28 amino acid octanoylated peptide that is produced by the stomach and which plays an important role in tissue growth and recovery from injury.

The term “alkyl” as used herein refers to straight chain, branched and cyclic fully saturated hydrocarbon residues, preferably straight chain or branched alkyl. Some non-limiting examples useful herein of straight and branched alkyl moieties of the invention include C1-20 alkyl, such as methyl, ethyl, propyl, isopropyl, butyl, isobutyl, see-butyl, tert-butyl, amyl, isoamyl, sec-amyl, 1,2-dimethylpropyl, 1,1-dimethylpropyl, hexyl, 4-methylpentyl, 1 methylpentyl, 2-methylpentyl, 3-methylpentyl, 1,1-demethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1,methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,2,2-trimethylbutyl, 1,1,3-trimethylbutyl, octyl, 6-methylheptyl, 1 methylheptyl, 1,1,3,3-tetramethylbutyl, nonyl, 1, 2, 3, 4, 5, 6, or 7-methyloctyl, 1, 2, 3, 4, or 5-ethylheptyl, 1,2, or 3-propylhexyl, decyl, 1, 2, 3, 4, 5, 6, 7 and 8-methylnonyl, 1, 2, 3, 4, 5, or 6-ethyloctyl, 1, 2, 3, or 4-propyllheptyl, undecyl, 1, 2, 3, 4, 5, 6, 7, 8 or 9-methyldecyl, 1, 2, 3, 4, 5, 6, or 7-ethylnonyl, 1, 2, 3, 4, or 5-propyloctyl, 1,2, or 3-butylheptyl, 1-pentylhexyl, dodecyl, 1, 2, 3, 4, 5, 6, 7, 8, 9, or 10-methylundecyl, 1, 2, 3, 4, 5, 6, 7, or 8-ethyldecyl, 1, 2, 3, 4, 5, or 6-propylnonyl, 1, 2, 3, or 4-butyloctyl or 1,2-pentylheptyl and the like.

Additional alkyl examples include C 21-25 alkyl, C 26-30 alkyl, C 31-35 alkyl, C 36-40 alkyl, C 41-46 alkyl, C 47-55 alkyl, C 56-60 alkyl, with examples of cyclic alkyls including mono- and polycyclic alkyl groups, such as cyclopropyl, cyclobutyl, cyclohexyl, cycloheptyl, cyclooctyl, cyclononyl, cyclodecyl and the like.

Further, as used herein, the term “alkenyl” refers to groups formed from straight chain, branched or cyclic hydrocarbon residues containing at least one carbon-carbon double bond, including ethylenically mono-, di-, or polyunsaturated alkyl or cycloalkyl groups as exemplified and defined above. Some representative examples of alkenyl groups or moieties include C1-20 alkenyl, such as vinyl, allyl, 1-methylvinyl, butenyl, iso-butenyl, 3-methyl-2-butenyl, 1-pentenyl, cyclopentyl, 1-methylcyclopentyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, and 1,3,5,7-cyclooctatetraenyl.

Additional examples of alkenyl groups include C 10-15 alkenyl, C 16-20 alkenyl, C 21-25 alkenyl, C 26-30 alkenyl, C 31-35 alkenyl, C 36-40 alkenyl, C 41-45 alkenyl, C 46-50 alkenyl, C 51-55 alkenyl, C 56-60 alkenyl, C 61-65 alkenyl, C 66-70 alkenyl, and C 71-80 alkenyl, with each of these examples possibly containing one or more alkyl or alkyl branches.

As employed herein, “substituted alkyl” comprises alkyl groups further bearing one or more substituents selected from hydroxyl, alkoxy (of a lower alkyl group), mercapto (of a lower alkyl group), cycloalkyl, substituted cycloalkyl, heteroaryl, aryloxy, substituted aryloxy, halogen, trifluoromethyl, cyano, nitro, nitrone, amino, amido, —C(O)H, acyl, oxyacyl, carboxyl, carbamate, sulfonyl, sulfonamide, sulfuryl, and the like.

As employed herein, “cycloalkyl” refers to cyclic ring-containing groups containing in the range of about 3 up to 8 carbon atoms, and “substituted cycloalkyl” refers to cycloalkyl groups further bearing one or more substituents as set forth above.

As employed herein, “heterocyclic” refers to cyclic (i.e., ring-containing) groups containing one or more heteroatoms (e.g., N, O, S, or the like) as part of the ring structure, and having in the range of 3 up to 14 carbon atoms and “substituted heterocyclic” refers to heterocyclic groups further bearing one or more substituents as set forth above.

As employed herein, “alkenyl” refers to straight or branched chain hydrocarbyl groups having a least one carbon-carbon double bond, and having in the range of about 2 up to 12 carbon atoms, and “substituted alkenyl” refers to alkenyl groups further bearing one or more substituents as set forth above.

As employed herein, “alkynyl” refers to straight or branched chain hydrocarbyl groups having at least one carbon-carbon triple bond, and having in the range of about 2 up to 12 carbon atoms, and “substituted alkynyl” refers to alkynyl groups further bearing one or more substituents as set forth above.

As employed herein, “aryl” refers to aromatic groups having in the range of 6 up to 14 carbon atoms and “substituted aryl” refers to aryl groups further bearing one or more substituents as set forth above.

As employed herein, “heteroaryl” refers to aromatic groups containing one or more heteroatoms (e.g., N, O, S, or the like) as part of the ring structure, and having in the range of 3 up to 14 carbon atoms and “substituted heteroaryl” refers to heteroaryl groups further bearing one or more substituents as set forth above.

As employed herein, “alkylaryl” refers to alkyl-substituted aryl groups and “substituted alkylaryl” refers to alkylaryl groups further bearing one or more substituents as set forth above.

As employed herein, arylalkyl” refers to aryl-substituted alkyl groups and “substituted arylalkyl” refers to arylalkyl groups further bearing one or more substituents as set forth above.

As employed herein, “arylalkenyl” refers to aryl-substituted alkenyl groups and “substituted arylalkenyl” refers to arylalkenyl groups further bearing one or more substituents as set forth above.

As employed herein, “arylalkynyll” refers to aryl-substituted alkynyl groups and “substituted arylalkynyl” refers to arylalkynyl groups further bearing one or more substituents as set forth above.

As employed herein, “aroyl” refers to aryl-carbonyl species such as benzoyl and “substituted aroyl” refers to aroyl groups further bearing one or more substituents as set forth above.

As employed herein, “acyl” refers to alkyl-carbonyl species.

As employed herein, “halogen” refers to fluoride, chloride, bromide or iodide atoms.

Generally, any compound possessing an amine terminal moiety and a carboxyl terminal moiety, obtained from any source, and which displays efficacy to any degree within a mammal for any reason is contemplated for use in this invention. Some preferred compounds are amino acids of the twenty or so compounds comprising the essential and non-essential groups, in addition to the amino acids taurine and citrulline Further, amino acids used herein may be natural, non-natural, or of natural extract origin, or a racemate, or an optically active material.

Several esters of amino acids are commercially available, and may be used in the present inventive supplement nutritional compositions, either individually or in mixtures. For example, amino acid ester compounds available from Varsal, Inc., include beta-Ala.OEt.HCl, Asp(OBzl)-OH, Asp(OBzl)-OBzl.TosOH, Cys.OEt.HCl, Glu(OBzl)-OH, Glu(OBzl).Tos.OH, Gin.OBzl.HCl, Gly.OMe.HCl, Gly.OBu.HCl, His.OMe.2HCl, Lys(L).OBzl.HCl, Phe-OBzl.HCl, Phe-OMe.HCl, Phe-OEt.HCl, Pro-OBzl.HCl, Ser-OMe.HCl, D,L-Ser.OMe.HCl, Trp-OMe.HCl, Tyr-OBu, Tyr-OMe.HCl, Tyr-OEt.HCl and Val-OMe.HCl (esters of alanine, aspartic acid, cysteine, glutamic acid, glutamine, ghycine, histidine, lysine, phenylalonine, praline, seriene, tryptophen, tyrosine and valine).

Other amino acids esters identified in wine which may be used herein are those reported in J. Agri. Food. Chem 1984, 32, 916-918. Ethyl esters of valine, leucine and isoleucine have also been detected in beer products. See, for example, J. Inst. Brewing 1981, 87, 85-86, which may also be used in the present compositions.

Additional esters contemplated for use herein include esters of alcohols and acids with one or more amino acids, including alkyl, alkenyl, and alkynal esters.

Some non-limiting examples of additional esters which may be used herein include methyl formate, propyl formate, isobutyl formate, butyl formate, methyl acetate, ethyl acetate, propyl acetate, isopropyl acetate, sec-butyl acetate, isobutyl acetate, butyl acetate, amyl acetate, sec-amyl acetate, iso-amyl acetate, sec-hexyl acetate, hexyl acetate, heptyl acetate, octyl acetate, methyl propionate, methyl butyrate, isobutyl isobutyrate, butyl butyrate, methyl methanoate, ethyl methanoate, butyl methanoate, methyl ethanoate, ethyl ethanoate, propyl ethanoate, 2-propyl ethanoate, butyl ethanoate, dimethyl ethyl ethanoate, methylpropyl ethanoate, pentyl ethanoate, 2 pentyl ethanoate, 3-methylbutyl ethanoate, hexyl ethanoate, 2-heptyl ethanoate, octyl ethanoate, 2-ethylhexyl ethanoate, nonyl ethanoate, methyl propanoate, ethyl propanoate, propyl propanoate, butyl propanoate, dimethyl ethyl propanoate, 3-methylbutyl propanoate, hexyl propanoate, and heptyl propanoate.

Some particularly preferred examples of compounds useful as nutrients and food supplements of the invention include, without limitation, arginine ethyl ester dihydrochloride, citrulline ethyl ester hydrochloride and glutamine ethyl ester hydrochloride.

If not obtained commercially, preparation of the amino acid esters of the invention may be accomplished by any conventional method, of which several are known. For example, U.S. Pat. No. 3,872,099 describes methods for producing active amino acid esters. Other examples are described in U.S. Pat. No. 5,113,009 for the preparation and isolation of a mineral acid salt of an amino acid methyl ester. In this example, a mineral acid is employed to form a salt with the amino group of the amino acid to function as a catalyst for esterification of the carboxylic acid group of the amino acid with an alkyl alcohol, such as methanol. Still other examples of amino acid ester synthesis may be found in EP 1044 676 and EPO 928 608 by Ajinomoto Co., which is also an excellent source for obtaining pharmaceutical grade amino acids, preferably in crystalline form for use in the present invention.

In a preferred embodiment, one or more or a mixture of amino acid esters as nutrient supplements of the invention may be orally administered to or by a user, for example, a person desirous of maintaining energy and promoting muscle and other body tissue development, or to combat fatigue, and to reduce muscle damage during exercise, or to enhance recovery after exercise, or for any end result contemplated. For example, a regimen for enhancing physical performance through amino acid nutritional amino acid supplements as disclosed in U.S. Pat. No. 5,026,721 may be used with the amino acid esters and peptide esters of this invention.

In an additional preferred embodiment, for example, the amino acid ester supplements of the invention may be administered orally individually, or in selected mixtures tailored to achieve a specific end result, as various and each of the essential and non-essential amino acids are known for their individual efficacy. For example, it has been reported that certain amino acids taken as isolated supplements may make up for a deficiency of that amino acid, or may make up for a shortage of chemicals from which an amino acid is synthesized, or for a shortage of molecules or chemicals that are made up from the amino acid. For instance, phenylalanine has been shown to relieve pain syndromes; tryptophan has been shown to raise serotonin levels in the brain to relieve depression; and tyrosine is known to help with depression as well. Glutamine may help balance blood sugar and regenerate the intestinal tract. Further, for example, isoleucine is known for its role with other branched amino acids in the rebuilding of muscle tissue, and in the release of energy, during muscular work, as is leucine. Lysine is known for its promotion of bone growth, and valine is noted for its role in muscle building. While these are merely some examples of the many efficacious effects of the known amino acids required in manuals, it can be seen that supplementing the supply of amino acids can help improve an assortment of different types of conditions in the body, individually or simultaneously.

In preferred embodiments, the amino acid ester supplements for use herein to enhance a mammals body performance, such as physical performance and the like, may comprise, individually, or alone, from about 0.001 grams to about 99.9 grams of the equivalent of glycine, or from about 0.001 weight % to about 80 weight % of a pharmaceutically acceptable carrier material;
from about 0.001 grams to about 99.9 grams of the equivalent of alanine, or from about 0.001 weight % to about 80 weight % of a pharmaceutically acceptable carrier;
from about 0.001 grams to about 99.9 grams of the equivalent of valine, or from about 0.001 weight % to about 80 weight % of a pharmaceutically acceptable carrier;
from about 0.001 grams to about 99.9 grams of the equivalent of leucine or from about 0.001 weight % to about 80 weight % of a pharmaceutically acceptable carrier;
from about 0.001 grams to about 99.9 grams of the equivalent of isoleucine or from about 0.001 weight % to about 80 weight % of a pharmaceutically acceptable carrier;
from about 0.001 grams to about 99.9 grams of the equivalent of phenylalamine or from about 0.001 weight % to about 80 weight % of a pharmaceutically acceptable carrier;
from about 0.001 grams to about 99.9 grams of the equivalent of proline or from about 0.001 weight % to about 80 weight % of a pharmaceutically acceptable carrier;
from about 0.001 grams to about 99.9 grams of the equivalent of serine or from about 0.001 weight % to about 80 weight % of a pharmaceutically acceptable carrier;
from about 0.001 grams to about 99.9 grams of the equivalent of threonine or from about 0.001 weight % to about 80 weight % of a pharmaceutically acceptable carrier;
from about 0.001 grams to about 99.9 grams of the equivalent of tyrosine or from about 0.001 weight % to about 80 weight % of a pharmaceutically acceptable carrier;
from about 0.001 grams to about 99.9 grams of the equivalent of cysteine or from about 0.001 weight % to about 80 weight % of a pharmaceutically acceptable carrier;
from about 0.001 grams to about 99.9 grams of the equivalent of methionine or from about 0.001 weight % to about 80 weight % of a pharmaceutically acceptable carrier;
from about 0.001 grams to about 99.9 grams of the equivalent of lysine or from about 0.001 weight % to about 80 weight % of a pharmaceutically acceptable carrier;
from about 0.001 grams to about 99.9 grams of the equivalent of arginine or from about 0.001 weight % to about 80 weight % of a pharmaceutically acceptable carrier;
from about 0.001 grams to about 99.9 grams of the equivalent of histidine or from about 0.001 weight % to about 80 weight % of a pharmaceutically acceptable carrier;
from about 0.001 grams to about 99.9 grams of the equivalent of tryptophan or from about 0.001 weight % to about 80 weight % of a pharmaceutically acceptable carrier;
from about 0.001 grams to about 99.9 grams of the equivalent of aspartic acid or from about 0.001 weight % to about 80 weight % of a pharmaceutically acceptable carrier;
from about 0.001 grams to about 99.9 grams of the equivalent of glutamic acid or from about 0.001 weight % to about 80 weight % of a pharmaceutically acceptable carrier;
from about 0.001 grams to about 99.9 grams of the equivalent of asparagine or from about 0.001 weight % to about 80 weight % of a pharmaceutically acceptable carrier;
from about 0.001 grams to about 99.9 grams of the equivalent of glutamine or from about 0.001 weight % to about 80 weight % of a pharmaceutically acceptable carrier; and
from about 0.001 grams to about 99.9 grams of the equivalent of taurine, or from about 0.001 weight % to about 80 weight % of a pharmaceutically acceptable carrier.

It is further contemplated that various amino acid esters of the invention be provided in peptide form. Peptide synthesis is well known, and specialty peptides may be provided on a made-to-order basis. See for example, Jerini Co. Any of such custom made peptides may be used as starting materials to form an amino-acid ester useful in the present invention.

In any event, the inventive compounds and nutritional supplements of the invention may be administered in therapeutically effective amounts, or that amount which provides therapeutic effects for a given condition and administration regimen. Further, the above examples are merely illustrative of some preferred compounds and their concentration ranges, and any amount of amino acid ester which displays any efficacy in any amount as a nutrient supplement for humans and animals for any indication, or is perceived to do so, such a placebo, may be employed in the invention.

Generally, without desiring to limit this invention to any theory, specific hypothesis, or biochemical synthetic mode of action, it is thought that the inventive compounds, at least with respect to oral administration, will be hydrolyzed at some point or location in a mammal's body and the hydrolysis products absorbed or adsorbed, or rejected as waste from the body. The rate of absorption will, of course, vary greatly from subject to subject, and absorption will occur at differing rates and in various combinations of different modes of action depending upon functionality. For example, some alkyl ester compounds of the invention are much less polar than the starting amino acid, with the ester functionality making these compounds intrinsically more lipophilic with decreased hydrogen bonding capacity and increased hydrocarbon content. Increased lipophilicity is a property which is consistent with enhanced membrane permeability and is often observed to improve absorption after oral administration. More particularly, it has been shown that some acyl ecdysteroids are liable to enzymic hydrolysis and may function as controlled release forms, or prodrugs, of the ecdysteroid. See, for example, Khimiya Rastitel'nogo Syr'ya, 2001, 2, 69-81 (Russian) (abstract), describing the chemical modification of 20-hydroxyecdysone and the study of membranotropic properties of its derivatives, and finding that the esterified products are liable to enzymic hydrolysis in vitro.

The compounds of the present invention, or at least their hydrolysis products, and their bio-activity and toxicity are for the most part well known, with amino acid containing food supplement compositions comprising a large share of the supplement dietary market.

As stated above, the amino acid esters may be used individually or in mixtures, as described above, or in free crystalline form or as pharmaceutically acceptable salts. They may also be used in any form with one or more pharmaceutically acceptable carriers, with adjuvants, excipients, or other essential nutrients, such as vitamins, minerals, electrolytes, carbohydrates and the like, or be used in combination with one or more pharmaceuticals for the treatment of any indication.

The phrase “pharmaceutically acceptable carrier” is recognized in the art, and includes a pharmaceutically acceptable material, composition or vehicle, suitable for administering in any way the compound(s) of the present invention to humans or animals. The carriers include, without limitation, liquid or solid filler, diluent, excipient, solvent or encapsulating material, involved in carrying the subject agent and/or compound(s), such as esterified amino acid ester, from one portion of the body, or organ, to another portion of the body, or organ. Each carrier must be “acceptable” in the sense of being compatible with the other ingredients of a formulation, and, of course, not injurious to the patient.

Some examples of materials which can serve as pharmaceutically acceptable carriers include, for instance, sugars, such as lactose, glucose, or sucrose; starches, such as corn starch and potato starch, cellulose and its derivatives, such as carboxymethyl cellulose, ethyl cellulose and cellulose acetate; natural and synthetic water soluble gums, such as powered tragacanth, guar gum and the like; malt; gelatin; talk; excipients, such as cocoa butter and suppository waxes; oils, such as peanut oil, palm oil, cottonseed oil, safflower oil, sesame oil, olive oil, corn oil, palm oil and soybean oil; glycols, such as propylene glycol; polyols, such as glycerine, sorbitol, mannitol and polyethylene glycol; natural and synthetic lipid emulsions, such as Interlipid, esters, such as ethyl oleate and ethyllaurate; agar and other gelling and/or thickening agents; pH adjusting and/or buffering agents, such as magnesium hydroxide, aluminum hydroxide and phosphate buffer solutions; alginic acid and salts thereof; saline; isotonic saline; ethyl alcohol; and other non-toxic compatible substances employed in pharmaceutical formulations.

Other agents and compounds which may be employed include, without limitation, wetting agents, emulsifiers and lubricants, such as sodium lauryl sulfate and magnesium stearate; coloring agents, release agents; coating agents; sweetening, flavoring and fragrance agents; preservatives and antioxidants, such as ascorbic acid, cysteine hydrochloride, sodium bisulfate, sodium meta bisulfite, sodium sulfite and the like. Some non-limiting examples of oil-soluble anti-oxidants useful herein include ascorbal palmitate, butylated hydroxyanisole (BHA), butylated hydroxytoluene (BHT), lecithin, propyl gallate and the like. Also useful herein are one or more metal chelating agents, such as citric acid, ethylenediamine tetraacetic acid (EDTA), sorbitol, tartaric acid, phosphoric acid and the like.

While preferred for oral deployment and ingestion, the amino acid esters and formulations thereof of this invention include those suitable for nasal, topical, transdermal, buccal, sublingual, rectal, vaginal, pulmonary and/or parenteral administration and/or may find use in any of an array of personal consumer body products, such as hair products, hand and body lotions, skin conditioners, and in treatments for finger and toenails.

Such formulations may be prepared by any conventional method(s) well known in the art of pharmacy, or by any non-conventional method, including convenient preparation by unit dosage form, with some exemplified methods including the step(s) of bring into association one or more compound(s), or amino acid esters, of the invention with one or more carriers and, optionally, one or more accessory ingredients, preferably uniformly and intimately, and if necessary, or desired, shaping the resulting product, and which will be administered by forms suitable for each administration route, such as by tablet and capsule form, by injection, inhalation, eye lotion, lotion or ointment, and suppository.

By “parenteral administration” and “administered parenterally” as used herein, means modes of administration other than enternal and topical administration, and usually by injection, which includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, aubarachnoid, intraspinal and intrasternal injection and infusion.

The terms “systemic administration” and “administered systemically”, “peripheral administration” and “administered peripherally” as used herein mean the administration of one or more of the inventive compound(s), such as amino acid esters, drugs or other material other than directly into the central nervous system, such that it enters a patient's or user's system/body and is subject to metabolism and similar processes, for example, such as subcutaneous administration and oral administration.

Subject to some preferred compounds and their preferred concentration ranges of use as mentioned above, the amount of active ingredient, be it amino acid ester and/or peptide ester or other bio-active ingredient, which can be combined with a carrier material to produce a single dosage form will generally be that amount effective to produce a therapeutic effect, or display any amount of efficacy, such as enhanced energy of the user, pain reduction and the like which shall generally referred to herein as a mammal effective nutritional supplement amount. In general, based upon one-hundred percent, this amount will range from less than about 0.0001 percent to more than about 99.999 percent of active ingredient, depending upon such variables such as the nature of the active ingredient employed, the user, the carrier material employed and the end result contemplated. Actual dosage levels of active ingredients in the inventive formulations may be varied so as to obtain an amount of the active ingredient(s) which is effective to achieve the desired end result contemplated or otherwise the desired therapeutic response (effective nutritional supplement amount) for a particular patient or user, composition, and mode of administration.

In another aspect of the invention, there are provided novel methods of conducting one or more business functions comprising the design, production, marketing, distribution, sale, licensing and/or leasing of the inventive compounds, or nutritional supplements formulations thereof and methods of treating and/or administrating same to person's and animals in need or desire thereof. The novel compounds, formulations and methods of the invention provide unique business opportunities heretofore unavailable, and which will enable the capture of a distinct and exclusive market share for its owners and licensees in the important health services sector with the invention's advantages over conventional methodology as described. It is further contemplated that the inventive subject matter herein be employed as a valuable business tool in the generation of business goodwill and as a vehicle for use in conjunction with trademarks to generate valuable source identifiers, and as subject matter to form and operate a business entity.

It is to be understood that many modifications and variations besides those preferred embodiments described above may be made in the inventive nutritional compositions of the invention without departing from the scope and spirit of invention and claims. It is also to be understood that the above embodiments are for illustrative purposes only and are not intended to limit the invention and for appended claims in any way.

1. A nutritional supplement comprising a compound of the formula: 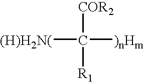
wherein R₁ is a biologically significant amino acid residue, or a naturally occurring amino acid residue, and R₂ is an ester of alcohols and acids comprising one or more of alkyl ester, alkenyl ester, alkynel ester, formates, acetates, acetoacetates, propionates, and esters of higher molecular weight monobosic acids, acrylates, methyl acrylates, esters of hydroxy acids and dibasic acids and esters of aromatic dibasic acids, and wherein said compound is present in a mammal active nutritional supplement amount; and where n is an integer from 1 to 20 and m is an integer from 1 to 40.

2. The nutritional supplement of claim 1 wherein R₁ is a residue of essential, non-essential and natural occurring amino acids, oligo-peptides thereof, and peptides thereof.

3. The nutritional supplement of claim 2 wherein R₁ is selected from the group consisting of glycine, alanine, valine, leucine, isoleucine, phenylalanine, proline, serine, threonine, tyrosine, cysteine, cystine, methionine, lysine, arginine, histidine, tryptophan, aspartic acid, glutamic acid, asparagine, glutamine, taurine, citrulline and gama amino butyric acid.

4. The nutritional supplement of claim 1 wherein said compound is present in amounts ranging from about 0.0001 weight % to about 99.999 weight % of the nutritional supplement.

5. The nutritional supplement of claim 1 which may be administered to mammals by way of one or more selected from the group consisting of oral, nasal, topical, transdermal, buccal, sublingual, rectal, vaginal, pulmonary and parenteral administration.

6. The nutritional supplement of claim 1 wherein said supplement further comprises a pharmaceutically acceptable carrier.

7. The nutritional supplement of claim 6 wherein said compound is present as a pharmaceutically acceptable salt.

8. The nutritional supplement of claim 6 further comprising one or more of additional bio-active compositions, vitamins, minerals, electrolytes, carbohydrates, pharmaceuticals and neutraceuticals.

9. A method for the production of the supplement of claim 1.

10. A method for the production of the supplement of claim 2.

11. A method for the production of the supplement of claim 3.

12. A method for the production of the supplement of claim 4.

13. A method for the production of the supplement of claim 5.

14. A method for the production of the supplement of claim 6.

15. A method for the production of the supplement of claim 7.

16. A method for the production of the supplement of claim 8.

17. A method for treating humans and/or animals in need thereof comprising and administering the nutritional supplement of claim 1.

18. A method for treating humans and/or animals in need thereof comprising and administering the nutritional supplement of claim 2.

19. A method for treating humans and/or animals in need thereof comprising and administering the nutritional supplement of claim 3.

20. A method for treating humans and/or animals in need thereof comprising and administering the nutritional supplement of claim 4.

21. A method for treating humans and/or animals in need thereof comprising and administering the nutritional supplement of claim 5.

22. A method for treating humans and/or animals in need thereof comprising and administering the nutritional supplement of claim 6.

23. A method for treating humans and/or animals in need thereof comprising and administering the nutritional supplement of claim 7.

24. A method for treating humans and/or animals in need thereof comprising and administering the nutritional supplement of claim 8.

25. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 1.

26. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 2.

27. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 3.

28. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 4.

29. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 5.

30. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 6.

31. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 7.

32. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 8.
